(19) United States
(12) Patent Application Publication
(10) Pub. No.: US 2003/0093225 A1
(43) Pub. Date: May 15, 2003

(54) Method for reducing noise in analytical assays

(76) Inventor: Hassan M. Fathallah-Shaykh, Chicago, IL (US)

Correspondence Address: Welsh & Katz, Ltd., Erik B. Flom, 22nd Floor, 120 South Riverside Plaza, Chicago, IL, 60606, US

(21) Appl. No.: 10/007,598

(22) Filed: Nov. 13, 2001

(51) Int. Cl. G06F 19/00; G01N 33/48; G01N 33/50; G01N 31/00

(52) U.S. Cl. 702/19; 702/20; 702/27

(57) ABSTRACT

A method of reducing noise in assay data collected in assaying measurables in a sample can provide replicate assay data for each of a plurality of measurables for one or more assay samples. The method can further provide a filtering function that identifies noise in replicate assay data. The method can also include a step of applying the filtering function to the replicate assay data to generate noise data. The method can also model the noise data to generate a noise model; and apply the noise model to the replicate assay data to reduce noise present in the replicate assay data.

### STATEMENT REGARDING FEDERALLY SPONSORED RESEARCH

This invention was made with United States government support under Grant Nos. R01-CA81367 and R29-CA78825 from the National Cancer Institute of the National Institutes of Health. The government of the United States has certain rights in the invention.

### BACKGROUND OF THE INVENTION

This invention generally relates to the analytical assay of physical properties of biological systems. In particular, the invention relates to the analysis of constituents in biological matter and the interactions among such constituents in analytical assays where the measurements are subject to a high proportion of noise relative to signal.

A number of advances in medicine, molecular biology, and genetics have led to increased demand for technologies that quantitatively measure properties of biological samples. The positive results of various genome mapping projects, including the Human Genome Project, have made increased research into gene-related fields. Accordingly, systems and methods for conducting measurements of gene expression levels, the abundance of RNA for encoding specific genes, protein expression levels, and other gene-related properties of biological matter have been in great demand.

In one response to the demand for conducting measurements of gene expression levels, gene expression profiling has emerged as a novel tool for rapid discovery of molecular expression patterns associated with human disease. (See Refs. 1-4)(References are listed at the end of the Detailed Description Of The Invention.) The completion of the first stage of the Human Genome Project has created the possibility of studying changes in gene expression of the complete genetic repertoire in any disease-affected tissue.

However, genome-wide screening is still hampered by the preponderance of false positive data in the gene microarray experimental system. (Ref 5) Such false positive data significantly impairs assessing which genes are significantly expressed in a cell, and what significant changes to such expression is occurring as cell conditions are varied. Although a large number of expressed sequence tags (ESTs) are known, many of the ESTs have no known function, have a falsely understood function, have a true known function but may have additional unknown functions, or may have known functions that are limited to certain conditions or cell types, but not known under other conditions or in other cell types.

Therefore there is a need for methods and systems that provide results for gene profiling experiments that are known to be true positive data to a high confidence level. It is desirable that the reproducibility of the results produced by such methods and systems be verifiable and verified by other technologies to instill confidence in the results. For example it would be desirable for the results of a method or system that identifies genes active in certain cell functions to be validated by what other investigators have reported using different paradigms for measuring molecular expression and by published functional biological experimental results. One desirable outcome for such methods and systems would be to provide data that link ESTs to cellular functions.

### BRIEF SUMMARY OF THE INVENTION

A method of reducing noise in assay data collected in assaying measurables in a sample can have the steps of:

providing replicate assay data for each of a plurality of measurables for one or more assay samples;

providing a filtering function that identifies noise in replicate assay data;

applying the filtering function to the replicate assay data to generate noise data;

modeling the noise data to generate a noise model; and

applying the noise model to the replicate assay data to reduce noise present in the replicate assay data.

In another aspect of the present invention, the filtering function can have filtering conditions, the filtering function being configured to operate on the replicate assay data to filter data based on the filtering conditions; where the filtering function is applied to the replicate assay data to designate the replicate assay data as being part of at least a first group and a second group, wherein the data in the first group satisfies the filtering conditions, and the data in the second group fails to meet at least one filtering condition.

In a still further embodiment of the present invention, the method further comprises the step of decomposing the second group to generate an eigenmatrix comprising a plurality of eigenvectors.

In another aspect of the present invention, modeling the noise data can comprise the steps of:

decomposing the noise data to generate decomposed noise data;

projecting the noise data onto the decomposed noise data to form projected noise data;

providing a model distribution having model distribution parameters; and

fitting the model distribution to the projected assay by calculating the model distribution parameters to generate a model noise distribution.

In yet another aspect of the present invention, the method of reducing noise in assay data of can further comprise:

providing a threshold eigendistance corresponding to the desired confidence level on the model noise distribution;

projecting the replicate assay data onto the eigenmatrix to generate replicate assay data eigendistances for each of the replicate assay data; and

selecting data from the replicate assay data having eigendistances greater than the threshold eigendistance;

wherein the replicate assay data having eigendistances greater than the threshold eigendistance are the significant data.

In another embodiment of the present invention, the replicate assay data are expression level measurements from a gene microarray experiment. Further, the filtering conditions comprise whether greater than a first percentage of the plurality of data for a given sample was manually adjusted, whether each of the plurality of data associated with an individual experimental sample has the same sign as each of the other data for that experimental sample, whether each expression level data for an experimental sample falls within a numerical range.

Another aspect of the present invention is a method of generating a filtering function for selecting significant data in assay data. Such a method can comprise the steps of:

providing a filtering function with at least one filtering parameter that can have a plurality of possible parameter values;

providing assay data comprising known false data;

evaluating the ability of the filtering function to remove false data from the assay data for a plurality of possible parameter values to generate respective filtering function effectiveness values;

using the filtering function effectiveness values to select a value for at least one filtering parameter of the filtering function to remove false data better than at least one other possible value of the filtering parameter.

In a further aspect of the present invention, the filtering parameter is the number of replicate measurements.

In still another aspect of the present invention, the number of replicate measurements can be about four to six replicate measurements.

In yet another aspect of the present invention the assay data are gene expression level measurements.

In another aspect of the present invention assay data comprising known false data can comprise:

providing a reference sample, wherein the reference sample generates a predominant majority of true positive reference results and a predominant minority of false negative reference results when studied with the experimental system;

providing a blank sample, wherein the blank sample generates a predominant majority of true negatives results with the experimental system;

providing an assay target sample, wherein the assay target sample generates no true positives when studied with the experimental system; and

studying the reference sample, blank sample and assay target sample with the experimental system, wherein the reference sample is used to generate true positive results, the blank sample is used to generate true negative results, and the assay target sample is used to generate false positive results,

wherein the true positive results, true negative results, and false positive results are used to select a value for the parameter of the filtering function from the possible parameter values that minimizes false positive results and false negative results.

Other features and advantages of the present invention will be apparent to those skilled in the art from the following detailed description, the accompanying drawings and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The benefits and advantages of the present invention will become more readily apparent to those of ordinary skill in the relevant art after reviewing the following detailed description and accompany drawings, wherein:

FIG. 1 illustrates the arrangement of spots on a 1.7 k gene microarray slide;

FIG. 2 illustrates a grid that can be used to define signal and background regions on an image of an illuminated arrangement of spots;

FIG. 3 graphically depicts the number of false positive and false negative readings not filtered by the function ƒₙ for various values of n for the exemplary embodiment;

FIG. 4 illustrates the relationships among the different E and N matrices;

FIG. 5 shows the distribution of the three most significant components of data known to be noise in the exemplary embodiment, FIG show the plots for N₁, N₂, N₃, and N₄ combined;

FIG. 6 shows the expression fractions of the various eigengenes in the assay of the exemplary embodiment;

FIG. 7 illustrates the 35th dimension eignendistances from the origin for each of the “noise” genes in the exemplary embodiment;

FIGS. 8A-D illustrate the 35th dimensional eigendistances from the origin for all of the genes in the exemplary assay, with FIG. 8A showing the known noise points from the N₁ matrix, FIG. 8B showing the data points from the E₁₁ matrix, FIG. 8C shows the distribution of the E matrix apart from the N₁ and E₁₁ matrices, and FIG. 8D shows the distribution of all be the gene's eigendistances;

FIGS. 9A-B are histograms showing the distribution of eigendistances for with FIG. 8A in FIG. 9A while the data points from FIG. 8B are shown in the in FIG. 9B;

FIG. 10 is a histogram showing the distribution of eigendistances for the points in FIG. 8C;

FIG. 11 is a color chart that shows data for the 35 samples of the exemplary assay for 92 significant genes including: the identities of the genes, the classifications of the tumors, the identified genetic classes of the genes, statistical analysis of the changes in regulation of the genes, the probability results of t-tests, and changes in expression level relative to the reference;

FIG. 12 is a color chart that shows data for the 35 samples of the exemplary assay for 16 less significant genes including: the identities of the genes, and changes in expression level relative to the reference;

FIG. 13 is a dendogram of the 35 samples used in the exemplary assay made by the Pearson method;

FIG. 14 is a dendogram of the 35 samples used in the exemplary assay made using the eigendistances among the exemplary assay samples to group the tumors; and

FIG. 15 is a color chart showing the variance of the replicate measurements of the genes in matrix C.

### DETAILED DESCRIPTION OF THE INVENTION

Although the present invention is susceptible of embodiment in various forms, there is shown in the drawings and will hereinafter be described presently preferred embodiments with the understanding that the present disclosure is to be considered an exemplification of the invention and is not intended to limit the invention to the specific embodiments illustrated.

It is to be further understood that the title of this section of the specification, namely, “Detailed Description of the Invention” relates to a rule of the U.S. Patent and Trademark Office, and is not intended to, does not imply, nor should be inferred to limit the subject matter disclosed herein or the scope of the invention.

Use of the present invention contemplates methods and systems for reducing noise in analytical assays. The present invention is illustrated with results of an exemplary analytical assay using a gene microarray system to analyze cDNAs in human gliomas. However, the teachings of the present invention can be applied to other analytical assays that are subject to false positive results or other forms of noise.

The present invention comprises systems and methods for providing a filtering function to reduce noise in analytical assays. The present invention also comprises, methods and systems using a filtering function to identify at least some of the noise in an analytical assay. The present invention further comprises methods and systems to model identified noise. The present invention also comprises methods and systems that use of a noise model of noise to reduce noise and select significant data.

One embodiment of the present invention contemplates assaying the contents of biological samples. A biological sample can be any cell, cell line, cell culture, tissue sample, organ, fluid or excretion of a living thing, including both plants and animals or other biological system recognized to those of ordinary skill in the art. Further, a biological sample can be an extract or derivative of a biological sample including, but not limited to complementary or copy DNA (cDNA), messenger RNA (mRNA), genomic DNA (gDNA), DNA, RNA, genes, gene fragments, chromosomes, single nucleotide polymorphisms (SNPs), oligonucleotides, proteins or any combination thereof An assayable quantity or quality can include measurements of structure, composition, or dynamics of a sample, and can be measured as a continuous basis or on a categorical basis. An example of a continuous measurement is to measure the fluorescence level of a probe molecule that has been attached to a species of interest. An example of a categorical measurement would be to detect whether a certain species is present in a sample, such as DNA sequence.

In a presently described embodiment, the systems and methods are for gene microarray assays. In the gene microarray assays, assay samples analyzed in comparison with reference samples. Blank or control samples can also be provided. An analytical assay of the present invention is directed to measuring a plurality of assayable quantities or qualities (measurables) of an assay sample. The results of measuring the assayable quantities or qualities of the sample are assay data. Similarly, measurements made of reference samples produce reference data, measurements made of blank samples produce blank data, and measurements made of control samples produce control data.

In the exemplary examples of the invention described in detail hereinafter, the microarray assays are directed to the analysis of assay samples comprising 35 glioma tumor samples that were obtained postmortem from consenting human individuals. Reference samples were obtained and pooled from four human individuals with no known neurological disease whose brains were frozen less than three hours postmortem. The tissues used in the assay samples and the reference sample were frozen in liquid nitrogen in the operating room. The quality of RNA in the tumor samples and the reference samples were determined by gel electrophoresis using methods known to those of ordinary skill in the art. Only high quality reference and sample RNAs were processed.

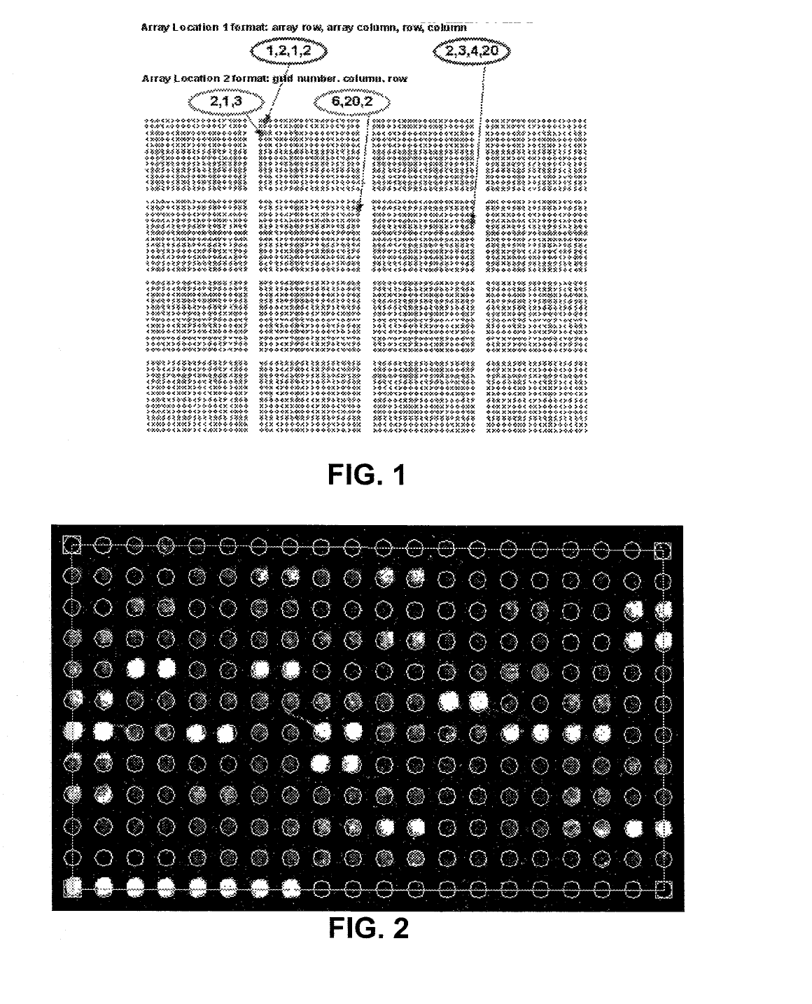

As illustrated in FIG. 1, microarray slides can have thousands of different DNA sequences arrayed in a defined matrix on a support, usually made of glass or silicon. Microarray slides suitable for use with the present invention are available The Microarray Centre at The Ontario Cancer Institute, University Health Network, Toronto, Ontario and other providers known to those of ordinary skill in the art. In the exemplary example of the present invention, total RNA from the samples and reference, preferably in the amount of 5-10 μg, can be reverse transcribed to yield cDNA. The sample and reference cDNA can then be labeled with a fluorescent probe by the amino-allyl method. Suitable fluorescent probes include cy3 and cy5 available from Amersham of Piscataway, N.J. The labeled cDNA can then be hybridized to the microarray slides via methods well known to those of ordinary skill in the art. Information regarding protocols for reverse transcription, fluorescent probe labeling and hybridization can be found at www.uhnres.utoronto.ca.

The slides containing the labeled cDNA can be scanned with suitable hardware and can optionally be visually analyzed also. One scanner suitable for use with the present invention is the 4000XL scanner available from Packard Bioscience of Meriden, Conn. Once scanned, images can analyzed with software available for a wide variety of computer platforms. Scanned images can be visually inspected and the data tagged, corrected, or otherwise evaluated individually with or without the aid of the imaging software. Imaging software suitable for use in the present invention Imagene Software available from Biodiscovery of Los Angeles, Calif.

In one embodiment of the present invention, Cy3 and Cy5 are used as fluorescent probes. Total RNA from samples can then be examined in two replicate experiments where the Cy3 and Cy5 probes were switched between sample the corresponding reference. For example in a first microarray run of the assay, the sample can be labeled with Cy3 and the reference sample Cy5, while in a second run of the assay, the sample is labeled with Cy5 and the reference sample Cy3. In order to increase the number of measurements per gene, microarray slides having duplicate spots are used. When microarray slides having two duplicate spots for each gene are used, this experimental design can generate four replicate data points for each per gene from a run of the assay.

Each of the spots in the exemplary assay can have a fluorescent signal intensity (or intensity) assigned to it. In the case of the microarray measurements, the intensity corresponds with the quantity of the probe that is present in a spot on the microarray by the binding of the labeled sample to the probe. The intensity for a given spot can be determined by calculating the background-subtracted mean intensities of corresponding normalized assay sample to reference sample ratios. Log2 values can be calculated for the ratios, with the log2 values that are positive, negative, or equal to 0 implying up regulation, down regulation, or no change in gene expression as compared to normal, respectively.

For the calculations that follow, bolded letters represent matrixes and vectors. * and ● denote multiplication and inner product, respectively. <b₁, . . . , bₙ> refers to the space defined by a basis {b₁, . . . , bₙ}. The mathematical computations detailed herein can be performed using functions written in Matlab provided by Mathworks of Natick, Mass. Examples of code that can be used for such are listed in the Appendices.

As will be understood by those of ordinary skill in the art, raw measurement data such as the fluorescent intensity can be the subject of a number of calculations to render a final data value for the measurement. Let G be the set of assay vectors, in the case of the exemplary gene microarray experiment being the set of genes being assayed. LetGTᵣ be the sets containing the replicate spots of the assay vectors (or genes) where r denotes the replicate number. Let A1 define ranges of valid final measurement results.

In the case of the exemplary gene microarray assay, the final measurement data is a referred to as a log2 number, but those of ordinary skill in the art will appreciate that there are other ways to express gene expression level, and that other experiments will have other types of final measurement data. The present invention can be useful for evaluating types of final measurement data other than log2 values. In the exemplary embodiment, the function prep: GTᵣ→A1 performs the following computations to provide log2 values. Code for carrying out the prep function is included in Appendix A. As will be appreciated by those skilled in the art, not all of the steps must be included, nor must all of the steps necessarily be performed in the order given.

1) Calculate the background subtracted mean intensity (BI) values of the reference (R) and assay sample (S). In the gene microarrray experiments of the illustrated example of the invention, the background is defined as the—mean background measured by the Imagene software for the scan at a given wavelength. As illustrated in FIG. 2, the Imagene software can be used to superimpose a grid that defines measured signal regions (circles) and background regions (squares encompassing the signal regions) for each spot. The measured signal intensity for a given spot is the intensity of the measurement inside the circle, while the background intensity for a given spot is the signal measured inside the box, but not within the circle. The background subtracted mean intensity is obtained by subtracting the intensity measured in the background region from the intensity measured in the signal region.

2) If data has been manually flagged as being undesirable, the background subtracted intensity values of all such manually flagged spots is changed to 0. Spots can be flagged manually when visual inspection reveals fluorescence caused by artifacts or dust or that the measurement has some other property that renders it undesirable.

3) For assays that use more than one pass, more than one sample or more than one slide, calculate a normalization factor. Such a normalization factor permits measurements of the same slide at different times, measurements of different samples, or the use of more than one slide to be related to one another. In the case of the microarray examples given below, the normalization factor of a slide is given as

$$NF = {\sum }_{w = 1 }^{i } {RBI}_{w} / {\sum }_{w = 1}^{i} {SBI}_{w} .$$

of every single microarray slide containing i spots (w refers to spots laid on a single slide).

4) Optionally, establish a minimum level for the background subtracted mean intensity and apply that to all measurements that have less than that level. In the case of the example embodiment, if BI<50 in one channel and >50 in the other (RBI or SBI), floor the value below 50 up to 50. In the equipment cited, a small number of 50 is used as a lower ceiling to avoid a denominator value that is near 0. Such small values often lead to erroneous results.

5) For each spot, calculate the ratio of the background corrected sample signal to the background corrected reference signal, Ratioᵣᵢ=SBIᵣᵢ/RBIᵣᵢ, where n and r refer to genes and the replicate number, respectively.

6) Optionally, establish upper and lower limits for the ratio of the sample and reference background corrected intensities. If Ratioᵣᵢ<0.02 or >50, truncate Ratioᵣᵢ to 0.02 or 50, respectively.

7) Calculate the normalized ratios, NRatioᵣᵢ=Ratioᵣᵢ*NF, where NF is the normalization factor of the slide where the spot ri is laid.

8) Calculate log₂ (NRatioᵣᵢ)

9) Let b and σ refer to the mean intensity and standard deviation of the local background, respectively; local background refers to the measurement by the Imagene software of the background immediately adjacent and surrounding the spot. Set the value of log₂(NRatioᵣᵢ) to 0, if the corresponding SBIᵣᵢ≦bᵣᵢ+2*σᵣᵢ and RBIᵣᵢ≦bᵣᵢ+2*σᵣᵢ, or SBIᵣᵢ−bᵣᵢ≦0 and RBIᵣᵢ−bᵣᵢ≦0.

The data of the 4 replicate spots processed by prep and the column vectors of the output are assembled to constitute the columns of the matrixes Uⱼ(iX2·r) corresponding to the assay sample number. For present notational purposes, which in no way limit the invention, odd numbered columns contain the log₂ data; and next higher numbered even numbered column designates whether the spot had been flagged manually.

Let G₁ᵣⱼ of size iXr denote the vectors containing the odd numbered columns of Uⱼ, associated with a replicate respectively. Let Qⱼ(i,r) be the matrix whose column r is G₁ᵣⱼ where j is the index associated with the assay sample number. Each gene row vector of Qⱼ contains the expression data of the r replicate spots; and j refers to the assay sample.

To study and model the noise in this experimental system we define the filtering functions ƒₙ for a plurality of n replicates of an assay vector, e.g. a row of Qⱼ(i,r) or its equivalent. An exemplary set of filtering functions of the present invention ƒₙ calculates the mean of the n replicate log2 values when the filtering conditions are met. However, as those of ordinary skill in the art will appreciate, the function ƒₙ can operate on values than a log2 values. It will also be understood by those of ordinary skill in the art, that the functions could be alternatively be defined in terms of failing to meet the filtering functions.

An exemplary set of filtering conditions for the set of filtering functions ƒₙ is: 1) all n log2 values are of the same sign and different than 0, 2) all n replicate ratios are within a specified replicate ratio range, and 3) at most 25% of the replicate values not flagged manually. If all 3 conditions are not met, ƒₙ calculates a value of 0. Preferably, the specified replicate ratio range is between <0.71 or >1.4 for a log2 replicate ratio. Code for implementing such a filtering function is provided in Appendix B.

The effectiveness of the filtering function at eliminating false results with a particular value of n can be determined. The present methods and systems can filter out false positive and false negative results. In the exemplary embodiment below, the exemplary assay described is especially prone to false positive results. Accordingly, the plurality of n replicate spots are analyzed with algebraic modeling of false positive data. However, it will be apparent to those of ordinary skill in the art how to use the same methods to treat systems prone to false negative data as well.

A process for studying whether ƒₙ correctly filters out false positive data without introducing false negative data can include the use of samples of material similar to that of the assay sample that are known to be unlike the assay samples and therefore unlikely to generate true positive data, but can generate false positive data. Such samples are referred to as control samples. Further, the process for evaluating whether ƒₙ can include assays of blank samples that are incapable of generating significant quantities of false positive data.

In an example of the present invention, the function ƒₙ is evaluated for an experimental system of RNA extracted from 35 glioma tumors mentioned previously. The reference sample is a sample comprising RNA from four healthy subjects. Microarray chips containing 1700 gene laid in duplicates (1.7K chips from the Ontario Cancer Institute) were provided. Each 1.7K chip contained a total of 128 spots of Arabidopsis cDNA with no known homology to human genes. To provide control samples, 64 spots of Arabidopsis cDNA were laid in duplicates to provide a total 128 spots. Further, 192 spots of buffer only (SSC) were laid out to provide 192 blank spots.

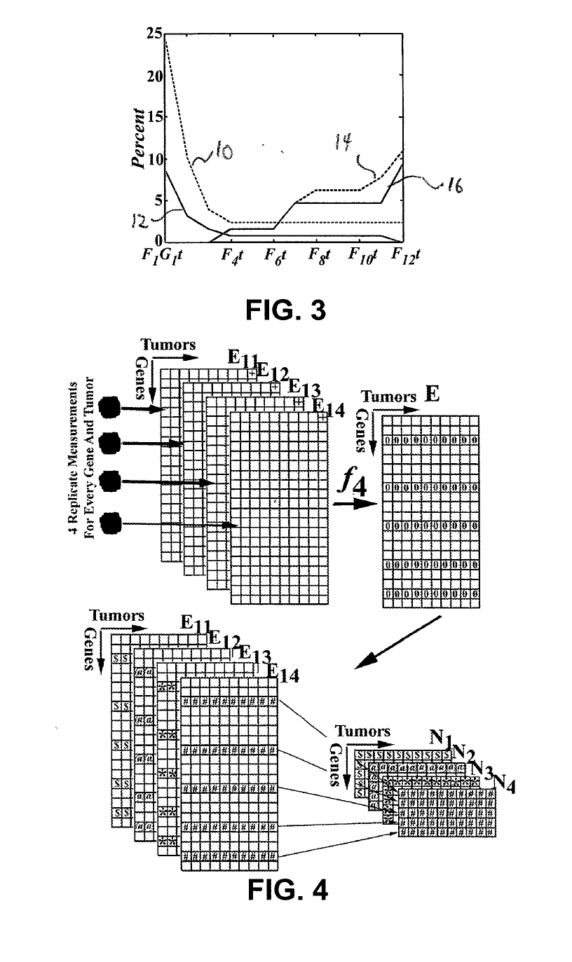

1 ng of Arabidopsis RNA transcribed in vitro was added to tumor RNA and either: 1) not added (dotted lines 10, 14), or 2) 0.5 ng added (solid lines 12, 16) to reference RNA. Each of these experiments was repeated 6 times to a total of 12 spots. The results of these experiments are shown in FIG. 3. The curves high on the left are false positive results 10, 12. The curves high on the right are false negative results 14, 16.

The results reveal that, after applying ƒ₄ to 4 replicate spots, 1.6% of the Arabidopsis spots are false negative, and 0-2% of the SSC spots without cDNA are false positive. Thus, ƒ₄ annuls false positive results without significant loss of data reflecting true changes in gene expression. As can be seen in FIG. 3, the same results were obtained for ƒ₆. However, as those of ordinary skill in the art will appreciate, assays using fewer replicates are less expensive than assays using more replicates, and is accordingly preferred for the exemplary embodiment.

The data to be analyzed in the exemplary embodiment of the invention was obtained from analyzing total RNA samples extracted from 35 human gliomas in reference to a single standard obtained by pooling RNA from human occipital lobes (reference RNA). The latter were harvested and pooled from 4 individuals with no known neurological disease whose brains were frozen less than 3 hours postmortem. Each glioma sample was analyzed on a 19K microarray consisting of 2 slides containing a total of 38400 spots representing 19200 genes laid in duplicates (19200 spots/slide). Total RNA (5-10 μg) was reverse transcribed and the cDNA products labeled by the amino-allyl method and hybridized to 19K gene microarrays purchased from the Ontario Cancer Institute (Toronto, Calif.). The slides were scanned at 10 μm by a confocal scanner, (4000XL scanner, Packard Bioscience; Meriden, Conn.). Images were analyzed by the Imagene Software (Biodiscovery; Los Angeles, Calif.). The data was analyzed as discussed above.

For the exemplary embodiment, let G be the set containing the 19200 genes. Let GTᵣ be the sets containing the replicate spots of the 19200 genes, 1≦r≦4; r refers to replicate number. The replicate number can be given by the desired level of quality for the noise filtering function. In the present exemplary example, given the results of the assay with the arabadiposis genes, a replicate number of 4 can be chosen to give desirable results. In the context of the data above, a replicate number of 6 would give equally good results, but be more expensive. However, the correct filtering conditions and number of replicate measurements can vary with the types of samples used and the behavior of those samples in a given assay. The false positives, true positives, false negatives, and true negatives generated by a sample in a given assay measuring a given set of vectors can vary as will be appreciated by those of ordinary skill in the art. The teachings of the above example, however, will be apparent to those of ordinary skill, who, without undue experimentation can select filtering conditions and the number of replicates balance the rates of false results against expense.

In the case of the present exemplary gene microarray experiments A1 is [−5.6,5.6]. The function prep: GTᵣ→A performs the above-detailed computations in sequence for the case of 19,200 spots on two slides each having 9600 spots. In the described embodiments the 19200 genes are evenly divided two slides, each slide having two spots of 9600 genes for a total of 19200 slots per slide, but only half the genes being present on a slide. E.g. the Ratioᵣᵢ=SBIᵣᵢ/RBIᵣᵢ, calculation is done for 1≦i≦19200, 1≦r≦4.

The predominant majority (>95%) of the data zeroed by ƒ₄ (applying the filtering function to 4 replicate measurements) in the assay are known to be false (see FIG. 3 and associated discussion above), therefore they can model the behavior of noise. A strategy similar to principal component analysis can project the gene vectors onto a space defined by linear transformation of their matrixes. It is preferred to model the noise matrixes by studying the projections of their gene vectors onto the space defined by all their eigenvectors, here the 35^{th} dimensional space, instead of projecting onto only the first few eigenvectors (principal components). However, use of less than all of the dimensions of the space can still be useful.

The filtering function separates data into at least two groups. In the filtering function ƒ₄ of the exemplary embodiment, the data is separated into two groups. Referring to FIG. 4, the unfiltered log2 values of the replicate spots were expressed in 4 matrixes E₁₁, E₁₂, E₁₃, E₁₄ of size (19200×35), each corresponding to one of the 4 replicate spots. The rows and columns refer to the 19,200 genes and 35 tumors, respectively. The filtered data after application of ƒ₄ were assembled to constitute a matrix E of size (19200×35); its rows correspond to the 19200 genes and columns to the 35 tumor samples. 9155 genes of E have log2 values=0 in all 35 tumor columns. One group of data created by the filtering function comprises the “noise” matrixes N₁, N₂, N₃, and N₄ are constructed to contain the unfiltered expression data of the 9155 genes mentioned above in E₁₁, E₁₂, E₁₃, and E₁₄, respectively.

The noise matrices can be decomposed via decomposition methods known to those of ordinary skill in the art. A suitable decomposition can be carried out using the “svd” function in Matlab. svd uses LAPACK routines to compute the singular value decomposition referring to Anderson, E., Z. Bai, C. Bischof, S. Blackford, J. Demmel, J. Dongarra, J. Du Croz, A. Greenbaum, S. Hammarling, A. McKenney, and D. Sorensen, LAPACK User's Guide, Third Edition, SIAM, Philadelphia, 1999.

Let ST_{J} be the set of row vectors (aᵢ₁,aᵢ₂,aᵢ₃,aᵢ₄)in Qⱼ, 1≦i≦19200, 1≦j≦35. Let A2 be [−5.6, −0.48]∪{0}∪[0.48,5.6]. The functions ƒₙ:ST_{J}→A2, 2≦n≦4, are defined by: ƒₙ((aᵢ₁,aᵢ₂,aᵢ₃,aᵢ₄))=0 if: 1) any aᵢₖε{aᵢ₁, . . . , aᵢₙ}, 1≦k≦n, has a different sign (+/−) than any other element in that set, 2) any −0.48≦aᵢₖ≦0.48, 1≦k≦n, (Please note that log₂(1.4)=0.48), or 4) more than 25% of the spots corresponding to {aᵢ₁, . . . , aᵢₙ} had been flagged manually. Otherwise, ƒₙ(aᵢ₁,aᵢ₂,aᵢ₃,aᵢ₄)=mean(aᵢ₁, . . . ,aᵢₙ). The data can be outputted into the vectors Kₙⱼ(19200×1).

Let E₁ᵣ(19200×35)be the matrixes whose jth columns vectors are G₁ᵣⱼ(19200×1), 1≦r≦4, 1≦j≦35. Let E (19200×35) be the matrixes whose jth columns vectors are K₄ⱼ(19200×1), 2≦n≦4. To model the noise, we created the matrixes Nᵣ, 1≦r≦4. Let (aᵢ₁, . . ,aᵢⱼ, . . ,aᵢ₃₅) denote the expression row vector of a gene gᵢ in F; and G₄ be the set of gene vectors in F such as gᵢεG₄ if aᵢⱼ≠0 for any 1≦j≦35. Nᵣ(9155×35) are generated by deleting from E₁ᵣ the expression rows corresponding to the genes in G₄, 1≦r≦4, respectively. The 4Nᵣ matrixes were linearly transformed by singular value decomposition,

Nᵣ(9155×35)=Uᵣ(9155×35)*Sᵣ(35×35)*Vᵣ^{T}(35×35) (0.1)

The columns of Vᵣ represent the eigengene vectors; S is a diagonal matrix containing the eigenvalues that reflect the “eigenexpression” levels or the amount of information carried by the corresponding eigengenes. The matrixes Vᵣ are orthogonal because

Vᵣ*Vᵣ^{T}=I and Vᵣ^{T}*Vᵣ=I (0.2)

I(35×35) is the identity matrix. Let {nᵣ₁, . . . ,nᵣ₃₅} be the sets containing the column vectors of Vᵣ, 1≦r≦4. (1.2) implies that {nᵣ₁, . . . , nᵣ₃₅} are orthonormal bases (eigenbases) of spaces defined by Vᵣ whose dimensions=35.

Let v and w be 2 vectors in space with angle θ between them, their inner product:

v●w=∥v∥*∥w∥*cos θ (0.3)

∥v∥=\sqrt{v●v} (0.4)

∥v∥ is the norm or length of v. ∥w∥*cos θ is the projection or coordinate (m) of w onto v. Therefore,

$$m = \frac{( v \cdot w )}{\sqrt{v \cdot v}} \qquad ( 0.5 )$$

The row vectors gᵢᵣ of Nᵣ, 1≦i≦19200, 1≦r≦4, were projected (to form eigenprojections) onto the 3-dimensional subspaces <nᵣ₁, nᵣ₂, nᵣ₃> of Vᵣ. Because ∥nᵣₖ∥=1, 1≦k≦35, the element at position (i, j) in the matrix PROJ:

PROJ=Nᵣ(9155×35)*Vᵣ(35×35)

represents the coordinate of the projection of the ith gene row vector of Nᵣ onto the kth eigengene of {nᵣ₁, . . . , nᵣₖ, . . . , nᵣ₃₅}. Projecting a row vector onto the eigenmatrix generates an eigendistance. The eigendistance from the origin of a vector v(m₁, . . . ,mₙ) in the nth dimensional space equals

$$\sqrt{{\sum }_{i = 1}^{n} {m}_{i}^{2}} .$$

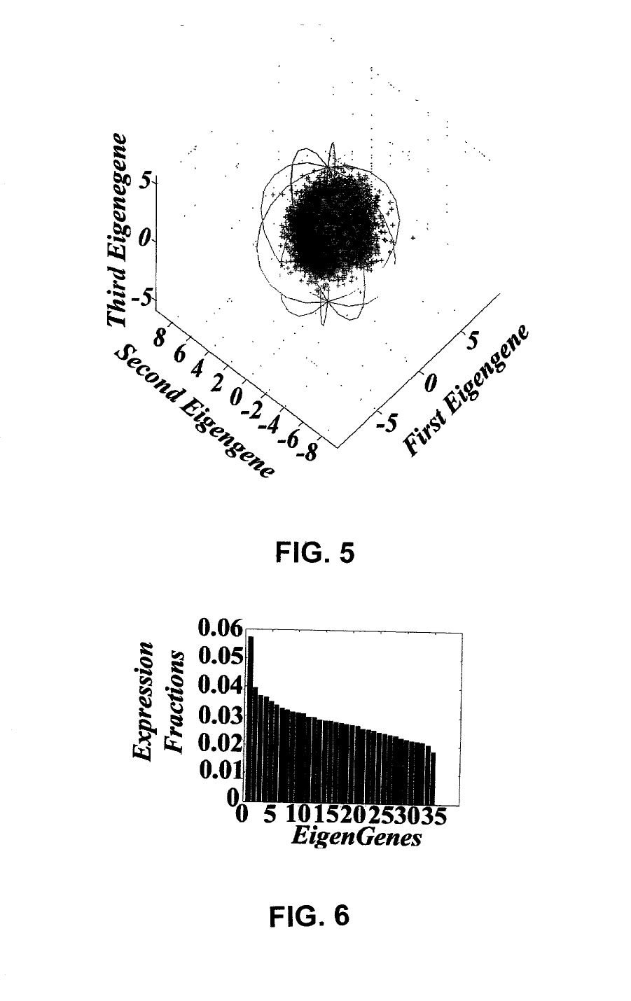

In the exemplary embodiment, the gene vectors of the 4 noise matrixes Nᵣ, 1≦r≦4, project within spherical structures in the 3^{rd} and 35^{th} dimensional eigengene spaces. FIGS. 5A-E are plots of the eigenprojections of the row vectors of N₁ (FIG. 5A), N₂ (FIG. 5B), N₃ (FIG. 5C), and N₄ (FIG. 5D) onto the subspaces defined by their corresponding first 3 eigengenes. FIG. 5E is a plot of the total projections of the noise matrices. As can be seen, the eigenprojections fall within a sphere around the origin.

Let evⱼ denote the eigenvalue corresponding the jth eigengene, 1≦j≦35. The expression fraction (EFⱼ) of the jth eigengene defined as:

$${EF}_{j} = {ev}_{j} / {\sum }_{j = 1}^{35} {ev}_{j} .$$

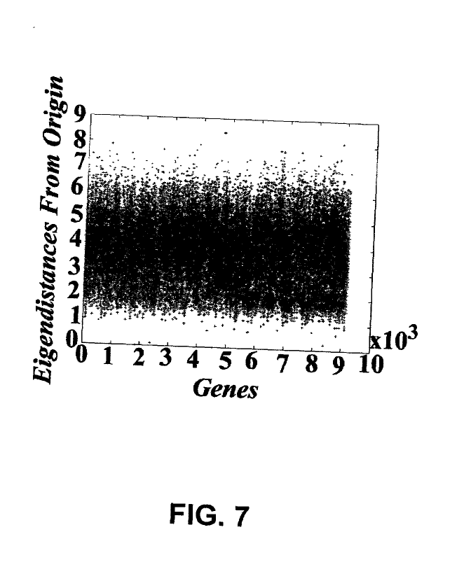

EFⱼ reflects the “amount of information” carried by the corresponding eigengenes. As can be seen in FIG. 6 the first three eigengenes capture 13.3%, 13.4%, 11.9%, and 11.9% of information in N₁, N₂, N₃, and N₄, respectively. Nonetheless, projecting the row vectors of Nᵣ, 1≦r≦4, onto their corresponding 35^{th} dimensional eigenspaces, as seen in FIG. 7, reproduces the spherical structure seen in the 3^{rd} dimensional space shown in FIG. 5E.

The invention can comprise selecting a model distribution and fitting the model distribution to the noise. Techniques known to those skilled in the art include visual inspection and testing the fit of alternative models to the data via a wide variety of techniques including least squares regression techniques. A useful tool for selecting possible distribution models is to plot histograms of the projections (eigen projections) of the row vectors of N₁, N₂, N₃, and N₄ onto the 35^{th} dimensional space.

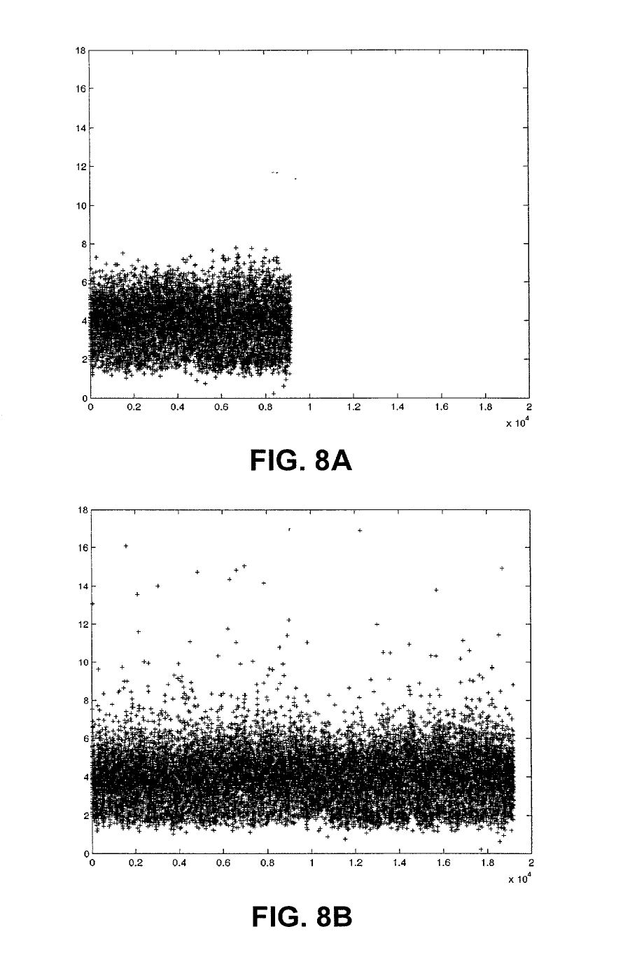

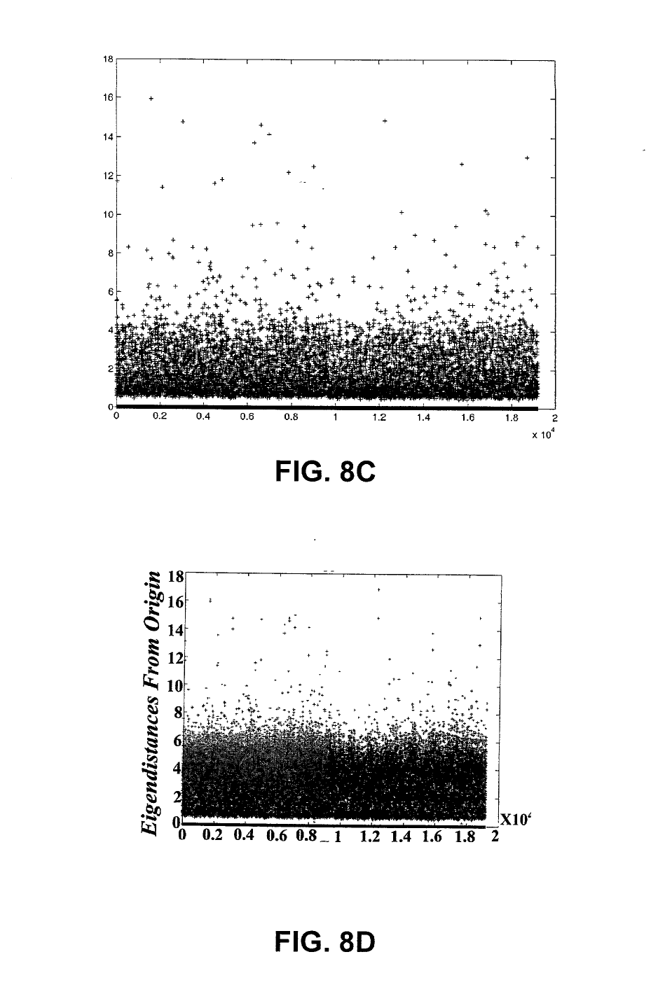

Referring to FIG. 8, a plot of the eigendistances of all of the gene vectors onto the 35^{th} dimensional spaces defined by their corresponding eigen vectors from the origin for the each of the genes is shown in a scatter plot. A first group of points, to the left of the dividing line, comprises the data from the noise matrices N₁, N₂, N₃, N₄, and a second group of points, to the right of the dividing line, comprises the data from the other data E₁₁, E₁₂, E₁₃, and E₁₄ are shown in a scatter plot.

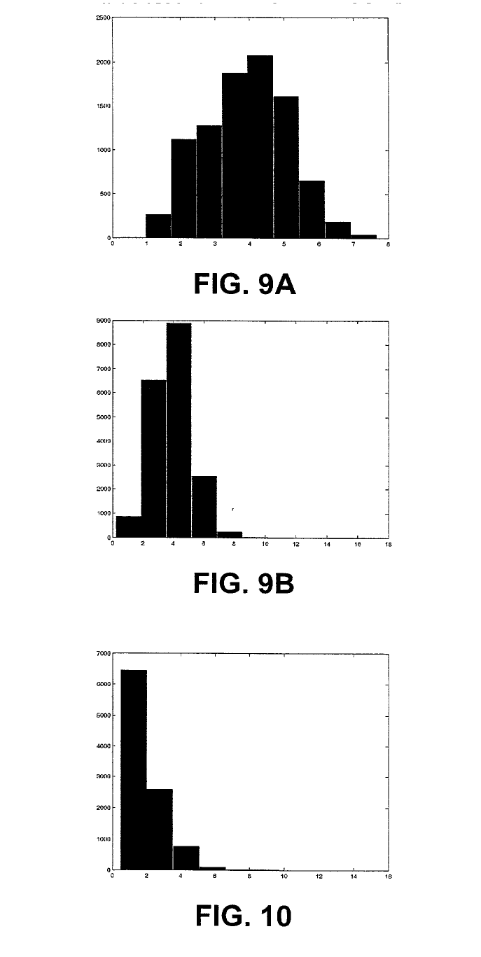

FIG. 9 shows histograms of the distances from the origin of the projections of row vectors of N₁ (lower plot) and E₁₁ (higher plot). Projections of the gene vectors of E reveal patterns similar to the projections of E₁₁ except that the former are closer to the origin. Referring to FIG. 10, a histogram plot of the eigendistances from origin of the gene vectors in E, excluding the ones that project at the origin, is also consistent with a normal distribution.

When a normal distribution is fit to the data from the noise matrices N₁, N₂, N₃, N₄, the result is a normal distribution having a standard deviation=1.2, and means=3.9, 3.9, 3.8, and 3.8, respectively. An existing Matlab function, normfit, can be used to accomplish the fit. Eigenprojections of the gene row vectors of E₁₁, E₁₂, E₁₃, E₁₄ onto their corresponding 35^{th} dimensional spaces reveals eigendistances that also follow normal distributions. When a normal distribution is fit to the eigendistances for the gene row vectors of E₁₁, E₁₂, E₁₃, E₁₄, means=4, 4, 3.9, 3.9, and standard deviations=1.4, 1.4, 1.3, 1.3, respectively were generated. A distance from the origin equal to 8.3 in the space defined by the eigenvectors of E₁₁ excludes the projections of genes vectors of the noise matrixes; this distance corresponds to a confidence level of >99.8% on the normal distribution curve defined by the fitted curve.

Accordingly, the method of the present invention can identify data that are very likely to be signal rather than noise. In the embodiment of the present exemplary assay, genes whose expression levels can be considered truly changed to the highest degree of certainty are identified. For the exemplary assay, projecting E onto the 35^{th} dimensional eigenspace defined by its eigenvectors reveals patterns similar to the projections of E₁₁, except that the eigendistances are closer to the origin. In addition, FIG. 8 shows that the 9155 gene vectors=0 of E project at the origin; by definition, these correspond to the same genes whose unfiltered data make up the noise matrixes.

When fit to a the normal distribution as a model, a histogram plot of the eigendistances to the origin of the genes in E, excluding those that project at the origin, is consistent with a normal distribution with a mean=1.9 and standard deviation=1.3 (FIG. 10). A confidence level that excludes genes that project farther than any noise vector (>99.8%) lends a higher degree of certainty than a confidence level of 99%.

Of the 19200 original gene vectors of E, 108 project at eigendistances>5.9 which corresponds to a confidence level>99.8% . The result is that 108 cDNAs have a confidence level of greater The expression levels of 92/108 cDNAs differ from normal brain in a minimum of 30% of the tumor set.

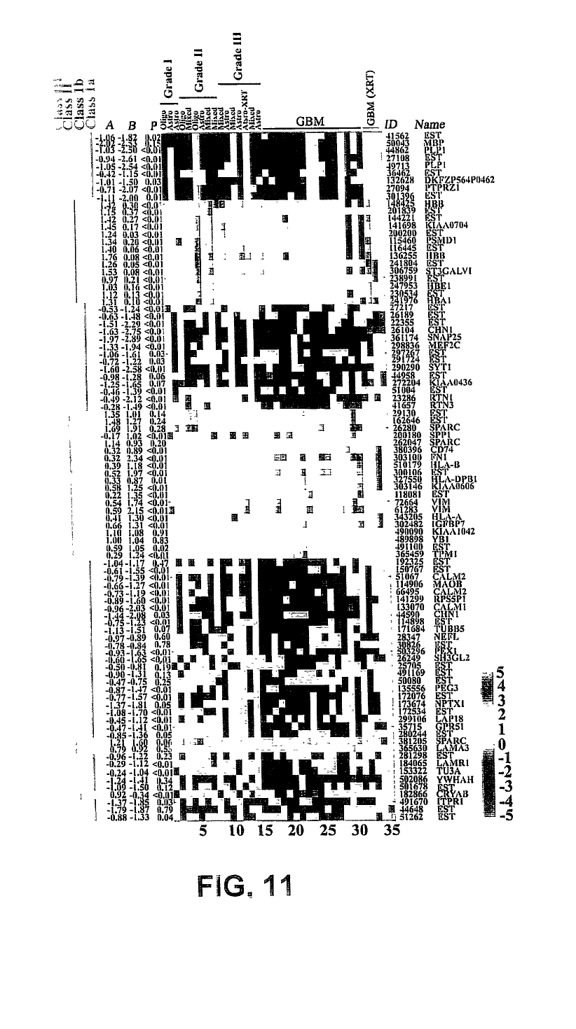

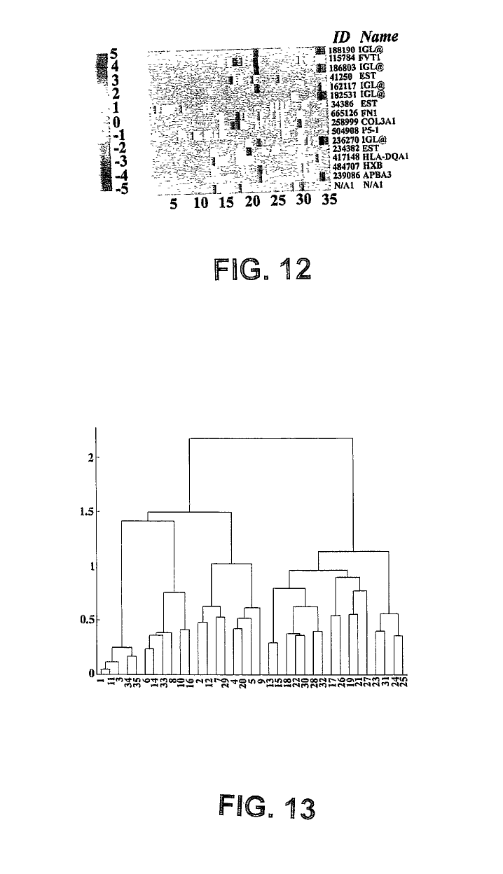

Once identified as being data that is significant to a very high level of confidence, assay data can be used to draw useful conclusions. In the exemplary assay, the 92 gene row vectors that varied in a minimum of 30% of the assay samples are first grouped by agglomerative hierarchical clustering using single linkage and their order re-arranged following their clusters to generate the matrix C (FIG. 11). The remaining 16 genes are shown in FIG. 12.

The assay samples in the exemplary assay were also evaluated using standard pathological techniques. The result of the evaluation was to assign standard accepted categories for each of the tumors based on widely accepted classifications. The World Health Organization's classification of brain tumors groups grades II and III tumors as anaplastic gliomas. Our tumor set contains 13 anaplastic gliomas (samples 4-16) and 17 GBM (samples 17-32). FIG. 11 reveals classes of genes that are up- and downregulated in gliomas and GBM as compared to normal brain. One can identify genes important for tumorigenesis whose expression levels differ between gliomas and GBM. To identify these genes and to capture all information in the expression data of the 4 replicate spots, the replicate data corresponding to the nonzero elements of C were compared between tumor samples 4-16 and 17-32 using a 2-sample t-test. Mean expression levels and p values are shown in FIG. 11. In FIG. 11, columns A and B refer to mean log₂ expression values in tumor samples 4-16 and 17-32, respectively. P is the p-value associated with the 2-sample t-test.

To show that molecular classification can replicate or surpass the standard pathological distinctions for classifying tumors, the tumor vectors in C were first grouped by agglomerative hierarchical clustering using Ward's incremental sum of squares of the 1-Pearson product moment correlation matrix. This first dendogram is formed using the 1-Pearson product moment correlation matrix

$$( 1 - \frac{\sum ( x - \overline{x} ) * ( y - \overline{y} )}{\sqrt{\sum {( x - \overline{x} )}^{2}} * \sqrt{\sum {( y - \overline{y} )}^{2}}} ) .$$

The result, the dendrogram shown in FIG. 13, reveals that glioblastoma multiforme (GBM) samples cluster together separate from lower grade tumors. Four high-grade glioma samples (14, 33, 34, & 35) that clustered with lower grade tumors showed pathological changes consistent with radiation necrosis.

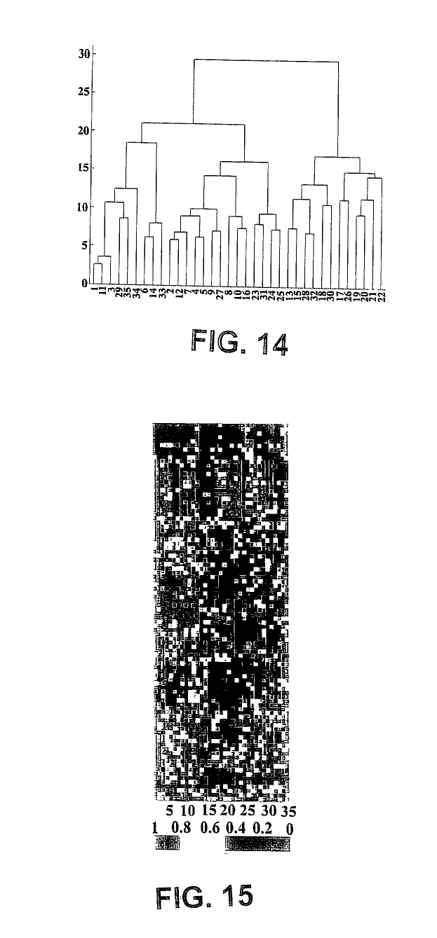

Rather than using standard techniques, a novel method of creating dendograms is provided by the present invention. This second method uses the distance matrix between eigenprojections in the 108^{th}-dimensional eigen space to render the dendogram of FIG. 14. This is done by measuring the distances between the endpoints of the row vectors of the genes in eigenspace to determine the grouping of the genes.

Let T be the matrix containing the expression of the 108 genes (columns) in the 35 tumors (rows). T(35×108) was transformed by singular value decomposition to yield the orthogonal matrix V_{T}(108×108) of dimension=108, defined by its eigenbasis {t₁, . . . , t₁₀₈}.

T(35×108)=U_{T}(35×108)*S_{T}(108×108)*V_{T}^{T}(108×108)

Therefore,

T*V_{T}=U_{T}*S_{T} (0.6)

The tumor row vectors of T are projected onto <t₁, . . . , t₁₀₈>. Because the 36^{th} to 108^{th} eigenvalues of S_{T} are equal to 0, the first 35 eigenvectors of {t₁, . . . , t₁₀₈} “carry 100% of the information.” In addition, (0.6) implies that the distance between the projections of any 2 tumor row vectors of T onto <t₁, . . . , t₁₀₈> is equal to the distance that separates their projections in <t₁, . . . , t₃₅>. The distance between 2 vectors v(v₁, . . . , v₁₀₈) and w(w₁, . . . , w₁₀₈) is:

$$‖ v - w ‖ = \sqrt{( v \cdot w ) \cdot ( v - w )} = \sqrt{{\sum }_{i = 1}^{108} {( {v}_{i} - {w}_{i} )}^{2}} \qquad ( 0.7 )$$

The dendrograms in FIGS. 13 and 14 are similar, showing that geometrical clustering of the tumors can generate results at least as good as those generated by prior techniques. The numbers in FIGS. 13 and 14 correspond to the tumor column vectors in FIG. 11. Samples 14, 33, 34, and 35 showed pathological changes consistent with radiation necrosis cluster with lower grade tumors. ID and Name refer to clone ID and name, respectively, and are clustered by both techniques.

To study the variance among the expression values of the 4 replicate spots, we created X(92×35×4), a 3-dimensional expression array, to contain the expression of the corresponding genes of C in E₁₁, E₁₂, E₁₃, and E₁₄. Let ƒv be the coefficient of variance:

$$fv = \vert \frac{s}{\overline{x}} \vert \qquad ( 0.8 )$$

s and \overline{x} refer to the standard deviation and mean, respectively. X permits computing Y(92×35), the matrix of coefficients of variance. The element at position (i, j) in Y is the coefficient of variance corresponding to the element at the same position as C.

In the exemplary assay, the coefficients of variance were computed to study the variance among the expression values of the 4 unfiltered replicate data corresponding to the elements of C (See FIGS. 11 and 15). Each element of FIG. 15 is the coefficient of variance of the 4 unfiltered replicate data that correspond to the element of C having the same matrix coordinates. FIGS. 11 and 15 demonstrate the high variance of the replicate data corresponding to the elements of C that were zeroed by ƒ₄, and the low variance of those that were not. Whereas the coefficient of variance of 1671/1671 and 16/1671 nonzero elements of C are <1 and ≧0.7, the coefficient of variance of 779/1512 and 1049/1512 zero elements are ≧1 and ≧0.7, respectively (FIG. 15). The expression patterns and identities of the gene vectors that project at higher than the 99% confidence are presented in FIG. 16 and Table I.

The methods of the present invention achieve results in a single, straightforward assay that until now required multiple laborious assays. As shown below, the results of previous narrow and expensive assays confirm many of the results of the exemplary assay. Further, using the methods and systems of the present invention, the exemplary assay provides sound data with a very high degree of confidence. The genes identified can be classified and used to understand how the cellular processes of gliomas differ from that of healthy occipital tissue. Such results can have immediate clinical application. For example, the ability to reliably recognize radiation necrosis with a gene microarray assay can provide important information that a clinician would want to have in weighing treatment options for a patient. Further, the results of the exemplary assay identify a number of cellular processes that would likely lead to improved treatments after appropriate clinical trials.

The results of the exemplary assay identify genetic expression classes that differ between anaplastic gliomas and glioblastomas and putative oncogenes and tumor suppressor genes in glial tumors. The genetic expression classes of these genes among these tumor types can be assigned with a high degree of certainty because the genes are known to have significantly different expression levels in gliomas to a high degree of certainty. Assigning the expression classes is possible because the experimental design of 4 replicate spots/gene combined with mathematical modeling assign a high degree of certainty that the measured changes in gene expression are true.

Patterns of genetic expression divide the 92-gene set shown in FIG. 11 into 3 classes. Class I includes genes downregulated in tumors as compared to normal brain. Expression levels of genes important for oligodendrocytes differentiation namely, myelin basic protein (MBP), proteolipid proteins (PLP), and protein tyrosine phosphatases (PTP), are downregulated in ⅔ grade I, 11/13 anaplastic tumors and 14/17 GBM (FIG. 11, class Ia).

Using in situ hybridization, Landry et al have reported findings similar to those of the exemplary assay (Ref. 6). Because the expression patterns of MBP, PLP, and PTP are tightly linked together to those of ESTs within a subclass identified by the clustering of the gene vectors (FIG. 11, class Ia) that the ESTs in class Ia are related to oligodendrocyte differentiation.

The exemplary assay of the present invention also shows that neuronal proteins and tumor suppressor genes are also downregulated in the tumor set (FIG. 11, class Ib). The latter includes neuronal proteins tyrosine 3-monooxygenase/tryptophan 5-monooxygenase activation protein (YWHAH), n-chimaerin (CHN1), synaptosomal-associated protein (SNAP-25), synaptotagmin I (SYT1), monoamine oxidase (MAO), calmodulin (CALM) I and II, and myocyte-specific enhancer-binding factor 2 (MEF2), the reticulon gene family (RTN1 and RTN3), peroxisomal matrix proteins (PEX1), beta tubulin (TUBB5), neuronal pentraxin (NPTX1), inositol triphosphate receptor (ITPR1), G-protein-coupled receptor (GPR51), neurofilament (NEFL1), stathmin (LAP18), SH3-containing Grb2-like (SH3GL2), and paternally expressed gene 3 (PEG3). Interestingly, PEG3 and SH3GL2 are thought to have tumor suppressor gene functions. PEG3 (19q13.4), induced by p53-mediated cell death processes, facilitates apoptosis by inducing translocation of Bax from cytosol to mitochondria. Furthermore, loss of heterogeneity of chromosome 19q is frequent in gliomas, and transfection of PEG3 cDNA into a glioma cell line abrogates tumorigenicity in nude mice (Refs. 7-9). Members of the SH3GL2 family are believed to couple signals from receptors and cytoplasmic tyrosine kinases to the Ras signaling pathway (Ref 10). TU3a (3p21.1) is a putative tumor suppressor gene located on the short arm of chromosome 3, a region commonly deleted in kidney, lung, breast, ovary, uterine, and head and neck cancers (Ref. 11).

Class II includes genes upregulated primarily in anaplastic tumors but significantly less so in GBM. Whereas hemoglobin alpha 1, epsilon 1 (11p15.5), and beta (11p15.5) are upregulated in more than 50% of anaplastic tumors, they are unchanged or down-regulated in more than 70% of GBM. Bianchi et al. reported complete loss of heterozygosity at the β-globin locus in 75% of mouse skin cancer. Furthermore, this was only detected in late-stage lesions exhibiting areas of dysplasia and microinvasion (Ref. 12). The authors postulated the presence of a putative tumor suppressor gene linked to the β-globin locus. Sialyltransferases (ST3GALVI) promote neuroblastoma growth and are correlated with tumor progression in non-small cell lung cancer (Ref 13, 14). The ubiquitin-proteasome has been correlated to malignant transformation by a variety of pathways including different sensitivities of isoforms of p53, p27, and c-Jun to degradation (Ref. 15). The proteasome 26s (PSMD1) is significantly upregulated in anaplastic gliomas (p<0.01). The results of Aoyama et al. (using Western blot) are similar to our findings that the low-molecular-weight heat shock protein crystalline alpha B (CRYAB) is upregulated in 70% of anaplastic tumors but not in most GBM (Ref 16).

Class III includes genes upregulated in both anaplastic tumors and GBM. Osteonectin (SPARC) is upregulated in more than 50% of anaplastic tumors and GBM. SPARC is a secreted glycoprotein widely distributed in tissue undergoing remodeling, morphogenesis, migration and proliferation. It interacts with extracellular matrix components, regulates matrix metalloproteinase expression, and stimulates angiogenesis. It is associated with neoplastic progression of human breast, colorectal cancers, and melanoma. Furthermore, down-regulation of SPARC by antisense RNA abrogates tumorigenicity of human melanoma cells (Ref 17-20). Osteopontin (SPP1) is found in all body fluids and in the proteinaceous matrix of mineralized tissue. It functions as a cell attachment protein and as a cytokine delivering signals by interacting with a number of receptors including integrins and CD44. Elevated osteopontin expression occurs in breast cancer, esophageal adenocarcinoma, and is positively correlated with tumor progression and worse prognosis in human lung adenocarcinoma and gastric tumors. The results of Saitoh et al using Northern blots and immunofluorescence are similar to our findings showing significant upregulation of osteopontin in GBM (p<0.01) (Refs. 21-24).

The elevated expression of MHC class I, (HLA-A, HLA-B, HLA-DPB1) and MHC class II (CD74) molecules (p<0.01), may be caused by tumor infiltrating lymphocytes. Components of the brain extracellular matrix including vimentin, fibronectin, and laminin are synthesized and secreted by astrocytes during development. Vimentin, previously reported by immunohistochemical analysis to be overexpressed in glial tumors, is an indicator of dedifferentiation and poor prognosis (p<0.01) (Ref 25). Fibronectin (FN1), a ligand for the integrin α5β1 promotes angiogenesis and tumor progression. Kochi et al and Higushi et al have shown fibronectin immunoreactivity in the blood vessels of proliferating gliomas (Refs. 26-28). Our results correlate fibronectin upregulation with progression to a higher malignant phenotype (p<0.01). Laminins have been implicated in a wide variety of biological processes including cell adhesion, differentiation, migration, signaling, neurite outgrowth and metastasis (Ref 29). FIG. 11 shows that Laminin 3 (LAMA3) is overexpressed in high-grade gliomas, but laminin receptor 1 (LAMR1) is downregulated in most GBM. The transcript of insulin growth factor-like binding protein (IGFB7; mac25) was initially cloned from leptomeningeal epithelial cells. Mac25 expression is upregulated in senescent human mammary epithelial cells and by treatment with retinoic acid, but is downregulated in breast carcinoma (Ref. 30). Paradoxically, our results show that mac25 is upregulated in anaplastic tumors and significantly more so in GBM (p<0.01). Tropomyosin (TPM1) isoforms form a family of rod-shaped proteins that bind to actin and are important for morphogenesis, neural differentiation, plasticity and formation of neuronal growth cones (Refs. 31, 32). The human Y-box binding proteins, YB-1, are transcription factors that are involved in a wide variety of biological functions including DNA repair, cell transcription, tumor resistance to cis-platinum, and interaction with p53 and large T antigen. YB-1 are overexpressed in almost all human colorectal cancerous lesions (Refs. 33-36). The results above implicate the ESTs of class III in oncogenesis and dedifferentiation to the malignant phenotype.

In brief, we report mathematical analysis and modeling of noise in the gene microarray experimental system, and describe formulas that allow separation of noise to a high degree of confidence. The methodology discovers genetic expression classes in gliomas and new putative oncogenes and tumor suppressor genes in glial tumors. While some of the results of the exemplary assay have been discovered before, no single prior experiment has been capable of reliably producing all of these results simultaneously. The methods and systems of the present invention

From the foregoing it will be observed that numerous modifications and variations can be effectuated without departing from the true spirit and scope of the novel concepts of the present invention. It is to be understood that no limitation with respect to the specific embodiment illustrated is intended or should be inferred. The disclosure is intended to cover by the appended claims all such modifications as fall within the scope of the claims.

### Reference List

1. Alter O, Brown, Botstein D. Singular value decomposition for genome-wide expression data processing and modeling. Proc Natl Acad Sci USA 2000; 97:10101-10106.

2. Golub T R, Slonim D K, Tamayo P, Huard C, Gaasenbeek M, Mesirov J P, et al. Molecular classification of cancer: class discovery and class prediction by gene expression monitoring. Science 1999; 286:531-537.

3. Alizadeh A A, Eisen M B, Davis E R, Ma C, Lossos I S, Rosenwald A, et al. Distinct types of diffuse late B-cell lymphomas identified by gene expression profiling. Nature 2000; 403, 503-511.

4. Bittner M, Meltzer P, Chen C, Jiang H, Seftor E A, Hendrix M, et al. Molecular classification of cutaneous melanoma by gene expression profiling. Nature 2001; 406, 536-539.

5. Lee M L T, Kuo F C, Whitmore G A, Sklar, J. Importance of replication in microarray gene expression studies: statistical methods and evidence from repetitive cDNA hybridizations. Proc Natl Acad Sci USA 2001; 97, 9834-9839.

6. Landry C, Verity M, Cherman L, Kashima T, Black K, Yates A, et al. Expression of oligodendrocytic mRNA in glial tumors: changes associated with tumor grade and extent of neoplastic infiltration. Cancer Res 1997; 57:4098-4104.

7. Deng Y, Wu X. Peg3/Pw1 promotes p53-mediated apoptosis by inducing Bax translocation from cytosol to mitochondria. Proc Natl Acad Sci USA 2000; 97:12050-12055.

8. von Deimling A, Louis D, von Ammon K, Petersen I, Wiestler O, Seisinger B. Evidence for a tumor suppressor gene on chromosome 19p associated with human astrocytomas, oligodendrogliomas, and mixed gliomas. Cancer Res 1992; 52:4277-4279.

9. Kohda T, Asai A, Kuroiwa Y, Kabayashi S, Aisaka K, Nagashima G, et al. Tumour suppressor activity of human imprinted gene PRG3 in a glioma cell line. Genes to Cells 2001; 6:237-247.

10. Feng G -S, Ouyang Y -B, Hu D -P, Shi Z -Q, Gentz R, Ni J. Grap is a novel SH3-SH2-SH3 adaptor protein that couples tyrosine kinases to the Ras pathway. J Biol Chem 1996; 271:12129-12132.

11. Yamato T, Orikasa K, Fukushige S, Orikasa S, Horii A. Isolation and characterization of the novel gene, TU3A, in a commonly deleted region on 3014.3→p14.2 in renal cell carcinoma. Cytogenet Cell Genet 1999; 87:291-295.

12. Bianchi A, Navone N, Aldaz C, Conti C. Overlapping loss of heterozygosity by mitotic recombination on mouse chromosome 7F1-ter in skin carcinogenesis. Proc Natl Acad Sci USA 1991; 88:7590-7594.

13. Hildbrandt H, Becker C, Gluer S, Rosner H, Gerardy-Schahn R, Rhamann H. Polysialic acid on the neural cell adhesion molecule correlates with expression of polysialyltransferases and promotes neuroblastoma cell growth. Cancer Res 1998; 58:779-784.

14. Tanaka F, Otake Y, Nakagawa T, Kawano Y, Miyahara R, Li M, et al. Expression of polysialic acid and STX, a human polysialyltransferase, is correlated with tumor progression in non-small cell lung cancer. Cancer Res 2000; 60:3072-3080.

15. Ciechanover A. The ubiquitin-proteasome pathway: on protein death and cell life. Embo J 1998; 24:7151-7160.

16. Aoyama A, Steiger R, Frohl E, Schafer R, von Deimling A, Wiestler O. 1993. Expression of αβ-crystallin in human brain tumors. Int J Cancer 1993; 55:760-764.

17. Sage H. Terms of Attachment: SPARC and tumorigenesis. Nat.Med. 1997; 3:144-146.

18. Ledda M, Adris S, Bravo A, Kairiyama C, Bover L, Chernajovsky Y, et al. Suppression of SPARC expression by antisense RNA abrogates the tumorigenicity of human melanoma cells. Nat.Med. 1997; 3:171-176.

19. Bellahcene A, Castronovo V. Increased expression of osteonectin and osteopontin, two bone marrow proteins, in breast cancer. Am J Path 1995; 146:95-100.

20. Porte H, De Moulins H, Gambiex L, Wurtz A, Quandalle P. Neoplastic progression of human colorectal cancer is associated with overexpression of the stromelysin-3 and BM-40/SPARC genes. Int J Cancer 1995; 64:70-75.

21. Dendhardt D, Giachelli C, Rittling S. Role of attachment of cellular signaling and toxicant injury. Annu Rev Pharmacol Toxicol 2001; 41:723-749.

22. Casson A G, Wilson S M, McCart J A, O'Malley F P, Ozcelik H, Tsao M S, el al. ras mutation and expression of the ras-regulated genes osteopontin and cathepesin in human esophageal cancer. Int J Cancer 1997; 72:739-745.

23. Ue T, Yokozaki H, Kitadai Y, Yamamoto S, Yasui Y, Ishikawa T, et al. Co-expression of osteopontin and CD44v9 in gastric cancer. Int J Cancer 1998; 79:127-132.

24. Saitoh Y, Kuratsu J, Takeshima H, Yamamoto S, and Ushio Y. Expression of osteopontin in human glioma. Its correlation with the malignancy. Lab Invest 1995; 72:55-63.

25. Yang H -Y, Lieska N, Shao D, Kriho V, Pappas G. Proteins of the intermediate filament cytoskeleton as markers for astrocytes and human astrocytomas. Mol Chem Neuropathol 1994; 21:155-176.

26. Kochi N, Tani E, Morimura T, Itagaki T. Immunohistochemical study of fibronectin in human glioma and meningioma. Acta Neuropathol 1983; 59:119-126.

27. Kim S, Bell K, Mousa S, Varner J. Regulation of angiogenesis in vivo by ligation of integrin a5b1 with the central-binding domain of fibronectin. Am J Path 2000; 156:1345-1362.

28. Higushi M, Ohnishi T, Arita N, Hiraga S, Hayakawa T. Expression of tenascin in human gliomas: its relation to histological malignancy, tumor dedifferentiation and angiogenesis. Acta Neuropathol 1993; 85:481-487.

29. Muir D, Johnson J, Rojiani M, Inglis B, Rojiani A, Maria B. Assessment of laminin-mediated glioma invasion in vitro and by glioma tumors engrafted within rat spinal cord. J Neuro-Oncol 1996; 30:199-211.

30. Ho C -L, Liem R. Enhanced expression of an insulin growth factor-like binding protein (mac25) in senescent human mammary epithelial cells and induced expression with retinoic acid. Proc Natl Acad Sci USA 1995; 92:4472-4476.

31. Stamm S, Casper D, Lees_Miller J, Hellman N. Brain-specific tropomyosins TMBr-1 and TMBr-3 have distinct patterns of expression during development and in adult brain. Proc Natl Acad Sci USA 1993; 90:9857-9861.

32. Schevzov G, Gunning P, Jefferey P, Temm-Grove C, Helfman D, Lin J, et al. Tropomyosin localization reveals distinct populations of microfilament in neurites and growth cones. Mol and Cell Neurosci 1997; 8:439-454.

33. Shibao K, Takano H, Nakayama Y, Okazaki K, Nagata N, Izumi H, et al. Enhanced coexpression of YB-1 and DNA topoisomerase II a genes in human colorectal carcinomas. Int J Cancer 1999; 83:732-737.

34. Okamoto T, Izumi H, Imamura T, Takano H, Ise T, Uchiumi T, et al. Direct interaction of p53 with the Y-box binding protein, YB-1: a mechanism for regulation of human gene expression. Oncogene 2000; 19:6194-6202.

35. Ise T, Nagatani G, Imamura T, Kato K, Takano H, Nomoto M, et al. Transcription factor Y-box binding protein 1 binds preferentially to cisplatin-modified DNA and interacts with proliferating cell nuclear antigen. Cancer Res 1999; 59:342-346.

36. Safak M, Gallia G, Ansari S, Khalili K. Physical and functional interaction between the Y-box binding protein YB-1 and human polyomavirus JC virus large T antigen. J Virol 1999; 73:10146-10157.

[image]

[image]

[image]

[image]

[image]

[image]

What is claimed is:

1. A method of reducing noise in assay data collected in assaying measurables in a sample comprising the steps of
providing replicate assay data for each of a plurality of measurables for one or more assay samples;
providing a filtering function that identifies noise in replicate assay data;
applying the filtering function to the replicate assay data to generate noise data;
modeling the noise data to generate a noise model; and
applying the noise model to the replicate assay data to reduce noise present in the replicate assay data.

2. The method of reducing noise in assay data of claim 1, wherein:
the filtering function has filtering conditions, the filtering function being configured to operate on the replicate assay data to filter data based on the filtering conditions; and
the filtering function is applied to the replicate assay data to designate the replicate assay data as being part of at least a first group and a second group, wherein the data in the first group satisfies the filtering conditions, and the data in the second group fails to meet at least one filtering condition.

3. The method of reducing noise in assay data of claim 2, further comprising the step of decomposing the second group to generate an eigenmatrix comprising a plurality of eigenvectors.

4. The method of reducing noise in assay data of claim 1, wherein modeling the noise data comprises the steps of:
decomposing the noise data to generate decomposed noise data;
projecting the noise data onto the decomposed noise data to form projected noise data;
providing a model distribution having model distribution parameters; and
fitting the model distribution to the projected assay by calculating the model distribution parameters to generate a model noise distribution.

5. The method of reducing noise in assay data of claim 3 further comprising:
providing a threshold eigendistance corresponding to the desired confidence level on the model noise distribution;
projecting the replicate assay data onto the eigenmatrix to generate replicate assay data eigendistances for each of the replicate assay data; and
selecting data from the replicate assay data having eigendistances greater than the threshold eigendistance;
wherein the replicate assay data having eigendistances greater than the threshold eigendistance are the significant data.

6. The method of claim 3, wherein:
the replicate assay data are expression level measurements from a gene microarray experiment; and
the filtering conditions comprise whether greater than a first percentage of the plurality of data for a given sample was manually adjusted, whether each of the plurality of data associated with an individual experimental sample has the same sign as each of the other data for that experimental sample, whether each expression level data for an experimental sample falls within a numerical range.

7. A method of generating a filtering function for selecting significant data in assay data comprising the steps of:
providing a filtering function with at least one filtering parameter that can have a plurality of possible parameter values;
providing assay data comprising known false data;
evaluating the ability of the filtering function to remove false data from the assay data for a plurality of possible parameter values to generate respective filtering function effectiveness values;
using the filtering function effectiveness values to select a value for at least one filtering parameter of the filtering function to remove false data better than at least one other possible value of the filtering parameter.

8. The method of claim 7, wherein the filtering parameter is the number of replicate measurements.

9. The method of claim 8, wherein the number of replicate measurements is about four to six replicate measurements.

10. The method of claim 7, wherein the assay data are gene expression level measurements.

11. The method of claim 7 wherein providing assay data comprising known false data comprises:
providing a reference sample, wherein the reference sample generates a predominant majority of true positive reference results and a predominant minority of false negative reference results when studied with the experimental system;
providing a blank sample, wherein the blank sample generates a predominant majority of true negatives results with the experimental system;
providing an assay target sample, wherein the assay target sample generates no true positives when studied with the experimental system; and
studying the reference sample, blank sample and assay target sample with the experimental system, wherein the reference sample is used to generate true positive results, the blank sample is used to generate true negative results, and the assay target sample is used to generate false positive results,
wherein the true positive results, true negative results, and false positive results are used to select a value for the parameter of the filtering function from the possible parameter values that minimizes false positive results and false negative results.

12. The method of claim 11, wherein the filtering parameter is the number of replicate measurements.

13. The method of claim 12, wherein the number of replicate measurements is about four to six replicate measurements.

14. The method of claim 11, wherein the assay data are gene expression level measurements, and the false positive results are measurements falsely show changes in gene expression, the false negative results are results failing to show changes in gene expression.
